SEQUENCE LISTING (2 sequences)

SEQ ID NO: 1 - DNA, 41 bp, Artificial Sequence
  Linker 1
caagttgggctttaaagcttgcagggcctgctgatgttgag

SEQ ID NO: 2 - DNA, 6 bp, Artificial Sequence
  Linker 2
aagctt

(19) United States
(12) Patent Application Publication
(10) Pub. No.: US 2022/0202863 A1
(43) Pub. Date: Jun. 30, 2022

(54) CELLS WITH MULTIPLEXED INHIBITORY RNA

(71) Applicant: CELYAD, Mont-Saint-Guibert (BE)

(72) Inventors: Simon BORNSCHEIN, Mont-Saint-Guibert (BE); Peggy SOTIROPOULOU, Mont-Saint-Guibert (BE); Eytan BREMAN, Mont-Saint-Guibert (BE); Mikhail STEKLOV, Mont-Saint-Guibert (BE)

(21) Appl. No.: 17/608,335

(22) PCT Filed: May 4, 2020
(86) PCT No.: PCT/EP2020/062346

§ 371 Date: Nov. 2, 2021

(30) Foreign Application Priority Data
May 2, 2019 (EP) 19172389.9

(51) Int. Cl. A61K 35/17; C12N 15/113; C07K 14/725; A61K 48/00

(52) U.S. Cl. CPC A61K 35/17; C12N 15/1138; C12N 2310/141; A61K 48/0058; C07K 14/7051

(57) ABSTRACT

The present application relates to the field of immunotherapy, more particularly to the field of adoptive cell therapy (ACT). Here, multiple shRNAs, designed to downregulate multiple targets are proposed. Also proposed are polynucleotides, vectors encoding the shRNA and cells expressing such shRNAs, alone or in combination with a chimeric antigen receptor (CAR). These cells are particularly suitable for use in immunotherapy.

### FIELD OF THE INVENTION

The present application relates to the field of immunotherapy, more particularly to the field of adoptive cell therapy (ACT). Here, multiple shRNAs, designed to downregulate multiple targets are proposed. Also proposed are polynucleotides, vectors encoding the shRNA and cells expressing such shRNAs, alone or in combination with a chimeric antigen receptor (CAR). These cells are particularly suitable for use in immunotherapy. The invention provides methods of increasing the efficacy of a T cell therapy in a patient in need thereof. Further, strategies to treat diseases such as cancer using these cells are also provided. The engineered immune cells, such as T-cells or natural killer (NK) cells, expressing such CARs are suitable for treating lymphomas, multiple myeloma and leukemia, but other tumors can be treated as well, depending on the specificity of the CAR.

### BACKGROUND

In cellular therapy, it is often advantageous to downregulate targets that could interfere with beneficial effects of the therapy: e.g. TCR components that could induce graft versus host disease, HLA components that could induce host versus graft disease, stress ligands, immune checkpoints, etc. However, it is often a problem to downregulate several of these targets simultaneously, both because of practical constraints (e.g. vector size and number of molecules that can be administered simultaneously), and because of toxicity.

Typically, genetic engineering approaches have been proposed, such as e.g. CRISPR/Cas, TALENs, zinc finger nucleases (ZFNs) and the like. However, these approaches typically lead to permanent and non-reversible changes and/or a complete knock-out of genes, which can be a problem if an absence of target leads to problems with viability or to toxicity. Furthermore, the permanent nature leads to less flexibility if only transient downregulation of a target is desired. Genetic engineering techniques typically are also quite cumbersome and are not ideally suited for simultaneous knockdown of several targets. E.g. in case of TALENs, for each target a separate nuclease protein needs to be engineered for a knockdown to be feasible. These then still need to be evaluated for efficacy. A combination of two or more different TALENs, while theoretically feasible, comes with clear practical disadvantages: the combination of the two still needs to be tested to check whether this has effects on efficacy. Since they are large proteins, it is impractical to express both in context of e.g. ACT, as vector size typically is limiting. All of these obstacles are exacerbated when more than two targets are considered. While Crispr/Cas typically is a more versatile solution, multiplexing (i.e. the simultaneous engineering of more than one target site) still proves challenging, particularly in eukaryotic organisms. This is caused by, amongst others, the low efficiency of DNA repair (the NHEJ repair mechanism of double strand breaks in eukaryotes is error-prone), the off-target effects and chromosomal rearrangements sometimes seen with CRISPR, and the generally low efficiency of multiplexing CRISPR (transfection efficiency dramatically decreases when more than one gene is targeted)—i.e. there are both problems with efficiency and specificity. Furthermore, gene editing approaches that permanently silence the targeted gene by acting directly to the DNA raise the requirement for robust testing to ensure genome integrity, while they also require elaborate, multi-step production methods, potentially leading to late differentiated or exhausted cells, with limited persistence and/or functionality (Gattinoni et al., 2011). This is particularly relevant in the context of ACT: for therapeutic efficacy, early differentiated and non-exhausted cells obviously are superior.

Thus, there is a need in the art to provide systems allowing cell therapy with multiplexed knockdown of targets that do not require multi-step production methods (and thus offer a comparative ease of manufacture and reduced costs), and offer flexibility (e.g. by making changes reversible, allowing attenuation of knockdown (e.g. to avoid toxicity), or swapping in one target for another).

### SUMMARY

When looking to solve the issues encountered with multiplexed genome engineering, systems could be considered that offer the possibility of a knockdown instead of a genetic knockout, which would lead to greater flexibility (e.g. temporal regulation would become possible). Ideally, these systems should also be less cumbersome (so that no separate proteins need to be engineered for each target), and should be sufficiently efficient and specific.

One solution that could be considered is RNA interference (RNAi). Several mechanisms of RNAi gene modulation exist in plants and animals. A first is through the expression of small non-coding RNAs, called microRNAs (“miRNAs”). miRNAs are able to target specific messenger RNAs (“mRNA”) for degradation, and thereby promote gene silencing.

Because of the importance of the microRNA pathway in the modulation of gene activity, researchers are currently exploring the extent to which small interfering RNAs (“siRNAs”), which are artificially designed molecules, can mediate RNAi. siRNAs can cause cleavage of a target molecule, such as mRNA, and similar to miRNAs, in order to recognize the target molecule, siRNAs rely on the complementarity of bases.

Within the class of molecules that are known as siRNAs are short hairpin RNAs (“shRNAs”). shRNAs are single stranded molecules that contain a sense region and an antisense region that is capable of hybridizing with the sense region. shRNAs are capable of forming a stem and loop structure in which the sense region and the antisense region form part or all of the stem. One advantage of using shRNAs is that they can be delivered or transcribed as a single molecule, which is not possible when an siRNA has two separate strands. However, like other siRNAs, shRNAs still target mRNA based on the complementarity of bases.

Many conditions, disease, and disorders are caused by the interaction between or among a plurality of proteins. Consequently, researchers are searching for effective ways to deliver multiple siRNAs to a cell or an organism at the same time.

One delivery option is the use of vector technologies to express shRNAs in the cells in which they will be processed through the endogenous miRNA pathway. The use of separate vectors for each shRNA can be cumbersome. Consequently, researchers have begun to explore the use of vectors that are capable of expressing a plurality of shRNAs. Unfortunately, the reported literature describes several challenges when expressing multiple shRNAs from a single vector. Among the issues that researchers have encountered are: (a) a risk of vector recombination and loss of shRNA expression; (b) reduced shRNA functionality by positional effects in a multiplex cassette; (c) the complexity of shRNA cloning; (d) RNAi processing saturation; (e) cytotoxicity; and (f) undesirable off-target effects.

Moreover, while siRNA has been shown to be effective for short-term gene inhibition in certain transformed mammalian cell lines, its use in primary cell cultures or for stable transcript knockdown proves more of a challenge. Knockdown efficacy is known to vary widely and ranges between <10% to >90% (e.g. Taxman et al., 2006), so further optimisation is necessary. As efficacy typically decreases when more than one inhibitor is expressed, this optimisation is even more important in such setting.

Therefore, there remains a need to develop efficient cassettes and vectors for delivery of multiplexed RNA interference molecules. While true for cellular applications in general, this is even less explored in the field of ACT, and there is a high need for efficient systems in these cells.

Surprisingly, it is demonstrated herein that not only shRNA can successfully be multiplexed in cells, particularly in engineered immune cells, but the targets are also very efficiently downregulated, even comparable to a genetic knockout (cf. Examples 5-8 and the comparison with CRISPR).

Accordingly, it is an object of the invention to provide engineered cells comprising a nucleic acid molecule encoding at least two multiplexed RNA interference molecules.

According to further embodiments, provided are engineered cells comprising:
A first exogenous nucleic acid molecule encoding a protein of interest
a second nucleic acid molecule encoding at least two multiplexed RNA interference molecules.

The engineered cells are particularly eukaryotic cells, more particularly engineered mammalian cells, more particularly engineered human cells. According to particular embodiments, the cells are engineered immune cells. Typical immune cells are selected from a T cell, a NK cell, a NKT cell, a stem cell, a progenitor cell, and an iPSC cell.

According to particular embodiments, the engineered cells further contain a nucleic acid encoding a protein of interest. Particularly, this protein of interest is a receptor, particularly a chimeric antigen receptor or a TCR. Chimeric antigen receptors can be directed against any target, typical examples include CD19, CD20, CD22, CD30, BCMA, B7H3, B7H6, NKG2D, HER2, HER3, GPC3, but many more exist and are also suitable.

According to specific embodiments, the first and second nucleic acid molecule are present in one vector, such as a eukaryotic expression plasmid, a mini-circle DNA, or a viral vector (e.g. derived from a lentivirus, a retrovirus, an adenovirus, an adeno-associated virus, and a Sendai virus).

The at least two multiplexed RNA interference molecules can be at least three, at least four, at least five, at least six, at least seven, at least eight, at least nine, at least ten or even more molecules, depending on the number of target molecules to be downregulated and the limitations of co-expressing the multiplexed molecules. A “multiplex” is a polynucleotide that encodes for a plurality of molecules of the same type, e.g., a plurality of siRNA or shRNA or miRNA. Within a multiplex, when molecules are of the same type (e.g., all shRNAs), they may be identical or comprise different sequences. Between molecules that are of the same type, there may be intervening sequences such as the linkers described herein. An example of a multiplex of the present invention is a polynucleotide that encodes for a plurality of tandem miRNA-based shRNAs. A multiplex may be single stranded, double stranded or have both regions that are single stranded and regions that are double stranded.

According to particular embodiments, the at least two multiplexed RNA interference molecules are under control of one promoter. Typically, this promoter is not a U6 promoter. This because this promoter is linked to toxicity, particularly at high levels of expression. For the same reason, one can consider to exclude H1 promoters (which are weaker promoters than U6) or even Pol III promoters in general (although they can be suitable in certain conditions). According to specific embodiments, the promoter is selected from a Pol II promoter, and a Pol III promoter. According to particular embodiments, the promoter is a natural or synthetic Pol II promoter. According to particular embodiments, the promoter is a Pol II promoter selected from a cytomegalovirus (CMV) promoter, an elongation factor 1 alpha (EF1α) promoter (core or full length), a phosphoglycerate kinase (PGK) promoter, a composite beta-actin promoter with an upstream CMV IV enhancer (CAG promoter), a ubiquitin C (UbC) promoter, a spleen focus forming virus (SFFV) promoter, a Rous sarcoma virus (RSV) promoter, an interleukin-2 promoter, a murine stem cell virus (MSCV) long terminal repeat (LTR), a Gibbon ape leukemia virus (GALV) LTR, a simian virus 40 (SV40) promoter, and a tRNA promoter. These promoters are among the most commonly used polymerase II promoters to drive mRNA expression.

According to particular embodiments, the at least two multiplexed RNA interference molecules can be shRNA molecules or miRNA molecules. Most particularly, they are miRNA molecules. A difference between shRNA molecules and miRNA molecules is that miRNA molecules are processed by Drosha, while conventional shRNA molecules are not (which has been associated with toxicity, Grimm et al., Nature 441:537-541 (2006)).

According to specific embodiments, the miRNA molecules can be provided as one miRNA scaffold under control of one promoter.

Particularly suited scaffold sequences for miRNA multiplexing are a miR-30 scaffold sequence, a miR-155 scaffold sequence, and a miR-196a2 scaffold sequence.

Typically, at least one of the miRNA molecules comprises a miR-196 scaffold sequence, preferably a miR-196a2 scaffold sequence. According to specific embodiments, all of the at least two miRNA molecules comprise a miR-scaffold sequence, preferably a miR-196a2 scaffold sequence. The same can be said for miR-30 and miR-155 scaffold sequences. Examples of such suitable scaffolds are listed in U.S. Pat. No. 8,841,267 (particularly claim 1 therein), incorporated herein by reference. The single scaffold is commercially available as the SMARTvector™ micro-RNA adapted scaffold (Horizon Discovery, Lafayette, Colo., USA). Multiple copies of this scaffold can be arranged in tandem repeats (see FIG. 5)

Further suitable scaffold sequences include miR-26b (hsa-mir-26b), miR-204 (hsa-mir-204), and miR-126 (hsa-mir-126), hsa-let-7f, hsa-let-7g, hsa-let-7a, hsa-let-7b, hsa-let-7c, hsa-mir-29a, hsa-mir-140-3p, hsa-let-7i, hsa-let-7e, hsa-mir-7-1, hsa-mir-7-2, hsa-mir-7-3, hsa-mir-26a, hsa-mir-26a, hsa-mir-340, hsa-mir-101, hsa-mir-29c, hsa-mir-191, hsa-mir-222, hsa-mir-34c-5p, hsa-mir-21, hsa-mir-378, hsa-mir-100, hsa-mir-192, hsa-mir-30d, hsa-mir-16, hsa-mir-432, hsa-mir-744, hsa-mir-29b, hsa-mir-130a, or hsa-mir-15a.

According to alternative, but not exclusive embodiments, rather than using a particular miR scaffold that is repeated, resulting in an artificially repeated scaffold, authentic polycistronic miRNA clusters or parts thereof can be used, where the endogenous miRNA is replaced by shRNA of interest. Particularly suitable miR scaffold clusters to this end are miR-106a^{˜}363, miR-17^{˜}92, miR-106b^{˜}25, and miR-23a^{˜}27a^{˜}24-2 cluster; most particularly envisaged is the miR-106a^{˜}363 cluster and fragments thereof. Of note, to save vector payload, it is also specifically envisaged to use part of such natural clusters and not all of the sequences (this is particularly useful as not all miRNAs are equally interspaced, and not all linker sequences may be needed). Other considerations can be taken into account, e.g. taking the miRNAs that are most efficiently processed in a cell. For instance, the miR-17^{˜}92 cluster consists of (in order) miR-17, miR-18a, miR-19a, miR-20a, miR-19b-1 and miR-92-1 (also miR-92a1), particularly useful fragments thereof are the scaffold sequence from miR-19a to miR-92-1 (i.e. 4 of the 6 miRNAs) or from miR-19a to miR-19b-1 (3 of the 6 miRNAs). Likewise, the 106a^{˜}363 cluster consists of (in order) miR-106a, miR-18b, miR-20b, miR-19b-2, miR-92-2 (also miR-92a2) and miR-363. Particularly useful fragments thereof are the scaffold sequence from miR-20b to miR-363 (i.e. 4 of the 6 miRNAs) or from miR-19b-2 to miR-363 (i.e. 3 of the 6 miRNAs). Both the natural linker sequences can be used, as well as fragments thereof or artificial linkers (again to reduce payload of the vectors).

It is envisaged that a combination of these strategies can be used, e.g. both the miR-106a^{˜}363 cluster and a miR-196a2 sequence can be combined in a novel scaffold.

According to particular embodiments, at least two of the multiplexed RNA interference molecules are directed against the same target. According to further specific embodiments, at least two of the multiplexed RNA interference molecules are identical.

According to alternative embodiments, all of the at least two multiplexed RNA interference molecules are different. According to further specific embodiments, all of the at least two multiplexed RNA interference molecules are directed against different targets.

Any suitable molecule present in the engineered cell can be targeted by the instant RNA interference molecules. Typical examples of envisaged targets are: a MHC class I gene, a MHC class II gene, a MHC coreceptor gene (e.g. HLA-F, HLA-G), a TCR chain, a CD3 chain, NKBBiL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCP5, DGKA, DGKZ, B2M, MICA, MICB, ULBP1, ULBP2, ULBP3, ULBP4, ULBP5, ULBP6, 2B4, A2AR, BAX, BLIMP1, C160 (POLR3A), CBL-B, CCR6, CD7, CD95, CD123, DGK [DGKA, DGKB, DGKD, DGKE, DKGG, DGKH, DGKI, DGKK, DGKQ, DGKZ], DNMT3A, DR4, DRS, EGR2, FABP4, FABP5, FASN, GMCSF, HPK1, IL-10R [IL10RA, IL10RB], IL2, LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, SHIP1, SOAT1, SOCS1, T-BET, TET2, TGFBR1, TGFBR2, TGFBR3, TIGIT, TIM3, TOX, and ZFP36L2.

Particularly suitable constructs have been identified which are miRNA-based. Accordingly, provided are engineered cells comprising a polynucleotide comprising a multiplexed microRNA-based shRNA encoding region, wherein said multiplexed microRNA-based shRNA encoding region comprises sequences that encode:

Two or more artificial miRNA-based shRNA nucleotide sequences, wherein each artificial miRNA-based shRNA nucleotide sequence comprises
a miRNA scaffold sequence,
an active or mature sequence, and
a passenger or star sequence, wherein within each artificial miRNA-based shRNA nucleotide sequence, the active sequence is at least 80% complementary to the passenger sequence.

Both the active sequence and the passenger sequence of each of the artificial miRNA-based shRNA nucleotide sequences are typically between 18 and 40 nucleotides long, more particularly between 18 and 30 nucleotides, most particularly between 19 and 25 nucleotides long.

Typically, these microRNA scaffold sequences are separated by linkers, and linker sequences can e.g. be between 30 and 60 nucleotides long, although shorter stretches also work. In fact, it was surprisingly found that length of linker plays no vital role and can be very short (less than 10 nucleotides) or even be absent without interfering with shRNA function. This is shown in FIGS. 6 and 16.

Artificial sequences can e.g. be naturally occurring scaffolds (e.g. a miR cluster or fragment thereof, such as the miR-106a^{˜}363 cluster) wherein the endogenous miR sequences have been replaced by shRNA sequences engineered against a particular target, can be repeats of a single miR scaffold (such as e.g. the miR-196a2 scaffold) wherein the endogenous miR sequences have been replaced by shRNA sequences engineered against a particular target, can be artificial miR-like sequences, or a combination thereof.

This engineered cell typically further comprises a nucleic acid molecule encoding a protein of interest, such as a chimeric antigen receptor or a TCR, and can be an engineered immune cell, as described above.

The co-expression of the multiplexed RNA interference molecules results in the suppression of at least one gene, but typically a plurality of genes, within the engineered cells. This can contribute to greater therapeutic efficacy.

The engineered cells described herein are also provided for use as a medicament. According to specific embodiments, the engineered cells are provided for use in the treatment of cancer.

This is equivalent as saying that methods of treating cancer are provided, comprising administering to a subject in need thereof a suitable dose of engineered cells as described herein, thereby improving at least one symptom.

The engineered cells may be autologous immune cells (cells obtained from the patient) or allogeneic immune cells (cells obtained from another subject).

### BRIEF DESCRIPTION OF THE FIGURES

FIG. 1: Optimization of miRNA scaffold length. A) Percentage of transduced T cells, as measured by CD19 expression. B) TCR and C) CD3E MFI of cells, transduced with a CD247 targeting shRNA, embedded in miRNA scaffolds of different length.

FIG. 2: Screening of different CD52 targeting shRNAs A) Percentage of transduced (CD19+) CD4+ or CD8+ T cells are shown, gated on FSC/SSC, viable, CD3+ cells. B) CD52 MFI is shown for transduced (gated in CD19+) CD4+ or CD8+ T cells. C) Representative histogram showing CD52 expression of transduced T (CD19+ CD3+) cells.

FIG. 3: CD52 knockdown in different donors. CD52 MFI is shown for T cells derived from three different donors. Cells were transduced with Mock or CD52 shRNA-3 expressing vector.

FIG. 4: Screening of gRNAs for the generation of CRISPR/Cas9 based CD52 knockout T cells. In the left panel, CD52 MFI is shown for CD4+ and CD8+ T cells at harvest (day 8). For Mock (tCD19) and shRNA condition, the gating was performed on CD19+ cells, whereas for the other conditions gating was performed on CD3+ T cells. In the right panel, representative histogram shows the CD52 expression for the three different gRNAs compared to Cas9 only control.

FIG. 5: Shows the design of CAR expression vector (e.g. CD19, BCMA, B7H3, B7H6, NKG2D, HER2, HER3, GPC3) without (top) or with (below) an integrated miRNA scaffold, allowing for the co-expression of a CAR and multiple shRNAs (e.g. 2, 4, 6, 8, . . . ) from the same vector. LTR: Long terminal repeat; promoter (e.g. EF1α, PGK, SFFV, CAG, . . . ); a marker protein (e.g. truncated CD34, CD19); multiplexed shRNAs.

FIG. 6: Two miRNA shRNAs were expressed from the same expression construct, in the context of a BCMA-CAR vector in primary T cells. Different spacers between the two shRNAs (multiplex 1-5) were assessed for their effect on knockdown of CD247 and CD52 protein. A) transduction efficiency measured by the expression of the reporter protein tCD34, B) BCMA CAR expression on the cell surface upon staining with a BCMA-Fc fusion protein, followed by a staining with an anti-Fc PE conjugated antibody. C) Mean fluorescence intensity (MFI) of TCR as a readout of the efficiency of downregulation of TCR expression upon knockdown of the CD3ζ subunit of the TCR mediated by the shRNA targeting CD247 and D) efficiency of the distinct constructs to downregulate CD52 using CD52 MFI as readout.

FIG. 7: A) CD247 (CD3z) and B) CD52 RNA levels were assessed by real-time PCR analysis, relative to CYPA RNA, used as housekeeping gene, in T cells transduced with the indicated single- or multiplexed shRNA constructs and respective controls.

FIG. 8: Representative flow cytometry data of TCR and CD52 stained T cells transduced with a BCMA CAR, co-expressing a CD247, a CD52 or both a CD52 and CD247 shRNA multiplexed with the spacer-2 or spacer-5. As a control, cells were nucleofected with an RNP Cas9 gRNA CD52 and gRNA CD247 complex.

FIG. 9: Flow cytometry analysis of A) TCR cell surface expression and B) CD52 cell surface expression of T cells transduced with the indicated single- or multiplexed shRNA constructs and respective controls.

FIG. 10: A) BCMA CAR expression of cells transduced with the different expression constructs was assessed by staining with BCMA-Fc fusion protein, followed by PE-conjugated anti-Fc and an APC-conjugated anti-CD34 antibodies. Median fluorescence intensity of BCMA-Fc staining is shown for transduced (CD34+) T cells. B) Different BCMA expressing cancer cell lines (RPMI-8226, U266, OPM-2) were co-cultured for 24 h with Mock (tCD34), BCMA-CAR expressing T cells with or without a CD247 shRNA, a CD52 shRNA or the CD247 CD52 multiplexed shRNA.), IFN-γ levels were measured in the supernatants by ELISA.

FIG. 11: Shows an in vitro functional assay of the T cell receptor in response to mitogenic stimuli. T cells were cultured in the presence of increasing concentrations an anti-CD3E antibody (clone OKT3). After 24 h the IFN-γ levels were measured in the supernatants by ELISA. Results from two different donors (CC19-174 and CC19-184) are presented.

FIG. 12: Shows an in vitro functional assay assessing the sensitivity of T cells to anti-CD52 mediated cell killing. Alemtuzumab was used as anti-CD52 antibody. T cells were treated with 30% complement in the presence of 50μg/mL alemtuzumab or IgG control antibodies. Number of viable cells was assessed after 4 h.

FIG. 13: Shows RNA expression in Jurkat cells transduced with four shRNAs targeting B2M, DGK, CD247 and CD52 expressed as single or multiplexed, as indicated on the vector design, along with a second generation CD19 CAR and a selection marker using a lentiviral backbone. Single step enrichment was performed using marker-specific magnetic beads on day 7 after transduction. shRNA-mediated downregulation of the transcriptional expression of the four targets was analyzed by qRT-PCR.

FIG. 14: Shown is RNA expression in primary T cells from a healthy donor transduced with retroviral vector encoding a second generation CD19-directed CAR, a truncated CD34 selection marker along with 3×sRNAs or 6×sRNAs targeting CD247, B2M or CD52, introduced in the 106a-363miRNA cluster. No shRNA (tCD34) was used as control. Two days after transduction, cells were enriched using CD34-specific magnetic beads, and further amplified in IL-2 (100 IU/mL) for 6 days. mRNA expression of CD247, B2M and CD52 was assessed by qRT-PCR using cyclophilin as house-keeping gene.

FIG. 15: Shows RNA expression in human iPSC cell line SCiPS-R1 transduced with two shRNAs separated by either long (linker 1-41 bp) or minimal (linker 2-6 bp) linker along with the selection marker CD34 (tCD34) using a lentiviral backbone. Transduction was performed with 50μl or 500μl of viral supernatant diluted till 1 ml total volume in culture medium. Single step enrichment was performed using CD34-specific CliniMACS magnetic beads on day 8 after transduction. Cells were subsequently analyzed for transcriptional expression of the shRNA targets by qRT-PCR, using cyclophilin as house-keeping gene. Bar graph represents relative expression values with SCiPS-R1 cells expressing no shRNA (tCD34) as control. Linker 1 (41 bp): caagttgggctttaaagcttgcagggcctgctgatgttgag (SEQ ID NO: 1); Linker 2 (6 bp—cloning derived): aagctt (SEQ ID NO: 2).

FIG. 16: Shows RNA expression in human iPSC cell line SCiPS-R1 transduced with two shRNAs separated by either long (linker 1-41 bp) or minimal (linker 2-6 bp) linker along with the selection marker CD34 (tCD34) using a lentiviral backbone. Transduction was performed with 500μl of viral supernatant diluted till 1 ml total volume in culture medium. Single step enrichment was performed using CD34-specific CliniMACS magnetic beads on day 8 after transduction. Cells were analyzed for shRNA targets expression by qRT-PCR using cyclophilin as house-keeping gene. Bar graph represents relative expression values with SCiPS-R1 cells expressing no shRNA (tCD34) as control.

### DETAILED DESCRIPTION

Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention.

Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

An “engineered cell” as used herein is a cell that has been modified through human intervention (as opposed to naturally occurring mutations).

The term “nucleic acid molecule” synonymously referred to as “nucleotides” or “nucleic acids” or “polynucleotide” as used herein refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “vector” is a replicon, such as plasmid, phage, cosmid, or virus in which another nucleic acid segment may be operably inserted so as to bring about the replication or expression of the segment. A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term “exogenous” as used herein, particularly in the context of cells or immune cells, refers to any material that is present and active in an individual living cell but that originated outside that cell (as opposed to an endogenous factor). The phrase “exogenous nucleic acid molecule” thus refers to a nucleic acid molecule that has been introduced in the (immune) cell, typically through transduction or transfection. The term “endogenous” as used herein refers to any factor or material that is present and active in an individual living cell and that originated from inside that cell (and that are thus typically also manufactured in a non-transduced or non-transfected cell).

“Isolated” as used herein means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

“Multiplexed” as used herein in the context of gene editing refers to the simultaneous targeting of two or more (i.e. multiple) related or unrelated targets. The term “RNA interference molecule” as used herein refers to an RNA (or RNA-like) molecule that inhibits gene expression or translation, by neutralizing targeted mRNA molecules. Examples include siRNA (including shRNA) or miRNA molecules. “Multiplexed RNA interference molecules” as used herein thus are two or more molecules that are simultaneously present for the concomitant downregulation of one or more targets. Typically, each of the multiplexed molecules will be directed against a specific target, but two molecules can be directed against the same target (and can even be identical).

A “promoter” as used herein is a regulatory region of nucleic acid usually located adjacent to a gene region, providing a control point for regulated gene transcription.

A “multiplex” is a polynucleotide that encodes for a plurality of molecules of the same type, e.g., a plurality of siRNA or shRNA or miRNA. Within a multiplex, when molecules are of the same type (e.g., all shRNAs), they may be identical or comprise different sequences. Between molecules that are of the same type, there may be intervening sequences such as the linkers described herein. An example of a multiplex of the present invention is a polynucleotide that encodes for a plurality of tandem miRNA-based shRNAs. A multiplex may be single stranded, double stranded or have both regions that are single stranded and regions that are double stranded.

A “chimeric antigen receptor” or “CAR” as used herein refers to a chimeric receptor (i.e. composed of parts from different sources) that has at least a binding moiety with a specificity for an antigen (which can e.g. be derived from an antibody, a receptor or its cognate ligand) and a signaling moiety that can transmit a signal in an immune cell (e.g. a CD3 zeta chain. Other signaling or cosignaling moieties can also be used, such as e.g. a Fc epsilon RI gamma domain, a CD3 epsilon domain, the recently described DAP10/DAP12 signaling domain, or domains from CD28, 4-1BB, OX40, ICOS, DAP10, DAP12, CD27, and CD2 as costimulatory domain). A “chimeric NK receptor” is a CAR wherein the binding moiety is derived or isolated from a NK receptor.

A “TCR” as used herein refers to a T cell receptor. In the context of adoptive cell transfer, this typically refers to an engineered TCR, i.e. a TCR that has been engineered to recognize a specific antigen, most typically a tumor antigen. An “endogenous TCR” as used herein refers to a TCR that is present endogenously, on non-modified cells (typically T cells). The TCR is a disulfide-linked membrane-anchored heterodimeric protein normally consisting of the highly variable alpha (α) and beta (β) chains expressed as part of a complex with the invariant CD3 chain molecules. The TCR receptor complex is an octomeric complex of variable TCR receptor α and β chains with the CD3 co-receptor (containing a CD3γ chain, a CD3δ chain, and two CD3ε chains) and two CD3ζ chains (aka CD247 molecules). The term “functional TCR” as used herein means a TCR capable of transducing a signal upon binding of its cognate ligand. Typically, for allogeneic therapies, engineering will take place to reduce or impair the TCR function, e.g. by knocking out or knocking down at least one of the TCR chains. An endogenous TCR in an engineered cell is considered functional when it retains at least 50%, at least 60%, at least 70%, at least 75%, at least 80%, or even at least 90% of signaling capacity (or T cell activation) compared to a cell with endogenous TCR without any engineering. Assays for assessing signaling capacity or T cell activation are known to the person skilled in the art, and include amongst others an ELISA measuring interferon gamma. According to alternative embodiments, an endogenous TCR is considered functional if no engineering has taken place to interfere with TCR function.

The term “immune cells” as used herein refers to cells that are part of the immune system (which can be either the adaptive or the innate immune system). Immune cells as used herein are typically immune cells that are manufactured for adoptive cell transfer (either autologous transfer or allogeneic transfer). Many different types of immune cells are used for adoptive therapy and thus are envisaged for use in the methods described herein. Examples of immune cells include, but are not limited to, T cells, NK cells, NKT cells, lymphocytes, dendritic cells, myeloid cells, stem cells, progenitor cells or iPSCs. The latter three are not immune cells as such, but can be used in adoptive cell transfer for immunotherapy (see e.g. Jiang et al., Cell Mol Immunol 2014; Themeli et al., Cell Stem Cell 2015). Typically, while the manufacturing starts with stem cells or iPSCs (or may even start with a dedifferentiation step from immune cells towards iPSCs), manufacturing will entail a step of differentiation to immune cells prior to administration. Stem cells, progenitor cells and iPSCs used in manufacturing of immune cells for adoptive transfer (i.e., stem cells, progenitor cells and iPSCs or their differentiated progeny that are transduced with a CAR as described herein) are considered as immune cells herein. According to particular embodiments, the stem cells envisaged in the methods do not involve a step of destruction of a human embryo.

Particularly envisaged immune cells include white blood cells (leukocytes), including lymphocytes, monocytes, macrophages and dendritic cells. Particularly envisaged lymphocytes include T cells, NK cells and B cells, most particularly envisaged are T cells. In the context of adoptive transfer, note that immune cells will typically be primary cells (i.e. cells isolated directly from human or animal tissue, and not or only briefly cultured), and not cell lines (i.e. cells that have been continually passaged over a long period of time and have acquired homogenous genotypic and phenotypic characteristics). According to specific embodiments, immune cells will be primary cells (i.e. cells isolated directly from human or animal tissue, and not or only briefly cultured) and not cell lines (i.e. cells that have been continually passaged over a long period of time and have acquired homogenous genotypic and phenotypic characteristics). According to alternative specific embodiments, the immune cell is not a cell from a cell line.

A “microRNA scaffold” or “miRNA scaffold” as used herein refers to a well-characterized primary microRNA sequence containing specific microRNA processing requirements, wherein a RNA sequence can be inserted (typically to replace existing miRNA sequence with a shRNA directed against a specific target). Examples of a miRNA scaffold include the SMARTvector™ micro-RNA adapted scaffold (Horizon Discovery, Lafayette, Colo., USA), or naturally occurring miRNA clusters such as the miR-106a^{˜}363 cluster.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In most particular embodiments of the described methods, the subject is a human.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

The phrase “adoptive cellular therapy”, “adoptive cell transfer”, or “ACT” as used herein refers to the transfer of cells, most typically immune cells, into a subject (e.g. a patient). These cells may have originated from the subject (in case of autologous therapy) or from another individual (in case of allogeneic therapy). The goal of the therapy is to improve immune functionality and characteristics, and in cancer immunotherapy, to raise an immune response against the cancer. Although T cells are most often used for ACT, it is also applied using other immune cell types such as NK cells, lymphocytes (e.g. tumor-infiltrating lymphocytes (TILs)), dendritic cells and myeloid cells.

An “effective amount” or “therapeutically effective amount” refers to an amount effective, at dosages and for periods of time necessary, to achieve a desired therapeutic result. A therapeutically effective amount of a therapeutic, such as the transformed immune cells described herein, may vary according to factors such as the disease state, age, sex, and weight of the individual, and the ability of the therapeutic (such as the cells) to elicit a desired response in the individual. A therapeutically effective amount is also one in which any toxic or detrimental effects of the therapeutic are outweighed by the therapeutically beneficial effects.

The phrase “graft versus host disease” or “GvHD” refers to a condition that might occur after an allogeneic transplant. In GvHD, the donated bone marrow, peripheral blood (stem) cells or other immune cells view the recipient's body as foreign, and the donated cells attack the body. As donor immunocompetent immune cells, such as T cells, are the main driver for GvHD, one strategy to prevent GvHD is by reducing (TCR-based) signaling in these immunocompetent cells, e.g. by directly or indirectly inhibiting the function of the TCR complex.

To assess whether the targeting of multiple genes in the context of adoptive cell transfer (ACT) is feasible without the need for genome editing (and its associated cost and complex manufacturing process), it was decided to test multiplexed RNA interference molecules.

The underlying approach is based upon the transcription of RNA from a specific vector that is processed by endogenous RNA editing machinery to generate an active shRNA which is able to target a mRNA of choice through base recognition and resultant destruction of that specific mRNA by the DICER complex. The specific destruction of the targeted mRNA results in the consequential reduction in expression of the relevant protein. Whilst RNA oligonucleotides can be transfected into target cells of choice to achieve a transient knockdown of gene expression, the expression of the desired shRNA from an integrated vector enables the stable knockdown of gene expression.

The successful expression of shRNA has largely been dependent upon coupling with a polymerase III (Pol III) promoter (e.g. H1, U6) that generate RNA species lacking a 5′ cap and 3′ polyadenylation, enabling processing of the shRNA duplex. Once transcribed, the shRNA undergoes processing, export from the nucleus, further processing and loading into the RNA-induced silencing complex (RISC) complex leading to the targeting degradation of mRNA of choice (Moore et al., 2010). Whilst effective, the efficiency of transcription driven by PolIII promoters can lead to cellular toxicity through the saturation of the endogenous microRNA pathway due to the excessively high expression of shRNA from PolIII promoters (Fowler et al., 2015). Moreover, expression of both a therapeutic gene and a shRNA by a single vector has been typically achieved through employing a polymerase II (PolII) promoter driving the therapeutic gene and a PolIII promoter driving the shRNA of interest. This is functional, but comes at the cost of vector space and thus offers less options for including therapeutic genes (Chumakov et al., 2010; Moore et al., 2010).

Embedding the shRNA within a microRNA (mir) framework allows the shRNA to be processed under the control of a PolII promoter (Giering et al., 2008). Importantly, the level of expression of an embedded shRNA tends to be lower, thereby avoiding the toxicity observed expressed when using other systems, such as the U6 promoter (Fowler et al., 2015). Indeed, mice receiving a shRNA driven by a liver-specific PolII promoter showed stable gene knockdown with no tolerability issue for more than one year (Giering et al., 2008). However, this was only for one shRNA, done in liver cells, and the reduction at protein level was only 15% (Giering et al., 2008), so it is not known whether higher efficiency can be achieved, also for more than one target, and particularly in immune cells (which are harder to manipulate).

Surprisingly, it is demonstrated herein that the expression of multiple microRNA-based shRNAs (based on e.g. the miR196a2 scaffold or miR106a^{˜}363 cluster used as scaffold) against different targets was feasible in T cells without showing recombination, without showing toxicity and while simultaneously achieving efficient downregulation of the targets.

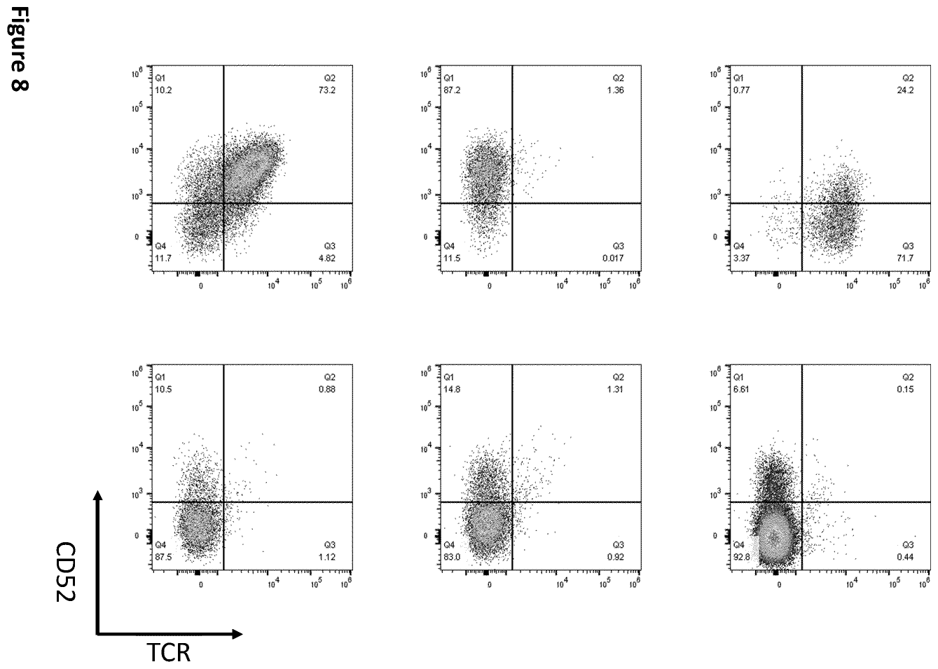

Thus, not only can shRNA be successfully multiplexed in cells, particularly in engineered immune cells, but the targets are also very efficiently downregulated, even comparable to a genetic knockout (cf. Examples 5-8 and FIGS. 8-12, providing a comparison with CRISPR).

Accordingly, it is an object of the invention to provide engineered cells comprising a nucleic acid molecule encoding at least two multiplexed RNA interference molecules.

Cells containing at least two RNA interference molecules can have advantages, particularly therapeutic benefits. RNA interference molecules can indeed be directed against targets of which (over)expression is undesirable. However, typically, the engineered cells provided herein will further contain a protein of interest.

According to these further embodiments, provided are engineered cells comprising:
a first exogenous nucleic acid molecule encoding a protein of interest, and
a second nucleic acid molecule encoding at least two multiplexed RNA interference molecules.

The optional further additional protein of interest can e.g. provide an additive, supportive or even synergistic effect, or it can be used for a different purpose. For instance, the protein of interest can be a CAR directed against a tumor, and the RNA interference molecules may interfere with tumor function, e.g. by targeting an immune checkpoint, directly downregulating a tumor target, targeting the tumor microenvironment. Alternatively or additionally, one or more of the RNA interference molecules may prolong persistence of the therapeutic cells, or otherwise alter a physiological response (e.g. interfering with GvHD or host versus graft reaction).

Proteins of interest can in principle be any protein, depending on the setting. However, typically they are proteins with a therapeutic function. These may include secreted therapeutic proteins, such as e.g. interleukins, cytokines or hormones. However, according to particular embodiments, the protein of interest is not secreted. Typically, the protein of interest is a receptor. According to further particular embodiments, the receptor is a chimeric antigen receptor or a TCR. Chimeric antigen receptors can be directed against any target expressed on the surface of a target cell, typical examples include, but are not limited to, CD5, CD19, CD20, CD22, CD23, CD30, CD33, CD38, CD44, CD56, CD70, CD123, CD133, CD138, CD171, CD174, CD248, CD274, CD276, CD279, CD319, CD326, CD340, BCMA, B7H3, B7H6, CEACAM5, EGFRvIII, EPHA2, mesothelin, NKG2D, HER2, HER3, GPC3, Flt3, DLL3, IL1RAP, KDR, MET, mucin 1, IL13Ra2, FOLH1, FAP, CA9, FOLR1, ROR1, GD2, PSCA, GPNMB, CSPG4, ULBP1, ULBP2, but many more exist and are also suitable. Although most CARs are scFv-based (i.e., the binding moiety is a scFv directed against a specific target, and the CAR is typically named after the target), some CARs are receptor-based (i.e., the binding moiety is part of a receptor, and the CAR typically is named after the receptor). An example of the latter is an NKG2D-CAR.

Engineered TCRs can be directed against any target of a cell, including intracellular targets. In addition to the above listed targets present on a cell surface, typical targets for a TCR include, but are not limited to, NY-ESO-1, PRAME, AFP, MAGE-A3, MAGE-A4, MAGE-A6, MAGE-A10, MAGE-Al2, gp100, MART-1, tyrosinase, WT1, p53, HPV-E6, HPV-E7, HBV, TRAIL, thyroglobulin, KRAS, HERV-E, HA-1, CMV, and CEA.

According to these particular embodiments where a further protein of interest is present, the first and second nucleic acid molecule in the engineered cell are typically present in one vector, such as a eukaryotic expression plasmid, a mini-circle DNA, or a viral vector (e.g. derived from a lentivirus, a retrovirus, an adenovirus, an adeno-associated virus, and a Sendai virus). According to further specific embodiments, the viral vector is selected from a lentiviral vector and a retroviral vector. Particularly for the latter vector load (i.e. total size of the construct) is important and the use of compact multiplex cassettes is particularly advantageous.

The engineered cells are particularly eukaryotic cells, more particularly engineered mammalian cells, more particularly engineered human cells. According to particular embodiments, the cells are engineered immune cells. Typical immune cells are selected from a T cell, a NK cell, a NKT cell, a stem cell, a progenitor cell, and an iPSC cell.

The at least two multiplexed RNA interference molecules can be at least three, at least four, at least five, at least six, at least seven, at least eight, at least nine, at least ten or even more molecules, depending on the number of target molecules to be downregulated and the limitations of co-expressing the multiplexed molecules. A “multiplex” is a polynucleotide that encodes for a plurality of molecules of the same type, e.g., a plurality of siRNA or shRNA or miRNA. Within a multiplex, when molecules are of the same type (e.g., all shRNAs), they may be identical or comprise different sequences. Between molecules that are of the same type, there may be intervening sequences such as linkers, as described herein. An example of a multiplex of the present invention is a polynucleotide that encodes for a plurality of tandem miRNA-based shRNAs. A multiplex may be single stranded, double stranded or have both regions that are single stranded and regions that are double stranded.

According to particular embodiments, the at least two multiplexed RNA interference molecules are under control of one promoter. Typically, when more than one RNA interference molecule is expressed, this is done by incorporating multiple copies of a shRNA-expression cassette. These typically carry identical promoter sequences, which results in frequent recombination events that remove the repeated sequence fragments. As a solution, typically several different promoters are used in an expression cassette (e.g. Chumakov et al., 2010). According to the present embodiments, however, recombination is avoided by the use of only one promoter. While expression is typically lower, this has advantages in terms of toxicity, as too much siRNA can be toxic to the cell (e.g. by interfering with the endogenous siRNA pathway). The use of only one promoter has the added advantage that all shRNAs are coregulated and expressed at similar levels. Remarkably, as shown in the Examples, multiple shRNAs can be transcribed from one promoter without a significant drop in efficacy.

According to further particular embodiments, both the at least two multiplexed RNA interference molecules and the protein of interest are under control of one promoter. This again reduces vector load (as no separate promoter is used to express the protein of interest), and offers the advantage of coregulated expression. This can e.g. be advantageous when the protein of interest is a CAR that targets a cancer, and the RNA interference molecules are intended to have an added or synergistic effect in tumor eradication.

Typically, the promoter used to express the RNA interference molecules is not a U6 promoter. This because this promoter is linked to toxicity, particularly at high levels of expression. For the same reason, one can consider to exclude H1 promoters (which are weaker promoters than U6) or even Pol III promoters in general (although they can be suitable in certain conditions). Thus, according to specific embodiments, the promoter used to express the RNA interference molecules is not a RNA Pol III promoter. RNA Pol III promoters lack temporal and spatial control and do not allow controlled expression of miRNA inhibitors. In contrast, numerous RNA Pol II promoters allow tissue-specific expression, and both inducible and repressible RNA Pol II promoters exist. Although tissue-specific expression is often not required in the context of the invention (as cells are selected prior to engineering), having specific promoters for e.g. immune cells is still an advantage, as it has been shown that differences in RNAi efficacy from various promoters were particularly pronounced in immune cells (Lebbink et al., 2011). According to specific embodiments, the promoter is selected from a Pol II promoter, and a Pol III promoter. According to particular embodiments, the promoter is a natural or synthetic Pol II promoter. Suitable promoters include, but are not limited to, a cytomegalovirus (CMV) promoter, an elongation factor 1 alpha (EF1α) promoter (core or full length), a phosphoglycerate kinase (PGK) promoter, a composite beta-actin promoter with an upstream CMV IV enhancer (CAG promoter), a ubiquitin C (UbC) promoter, a spleen focus forming virus (SFFV) promoter, a Rous sarcoma virus (RSV) promoter, an interleukin-2 promoter, a murine stem cell virus (MSCV) long terminal repeat (LTR), a Gibbon ape leukemia virus (GALV) LTR, a simian virus 40 (SV40) promoter, and a tRNA promoter. These promoters are among the most commonly used polymerase II promoters to drive mRNA expression.

According to particular embodiments, the at least two multiplexed RNA interference molecules can be shRNA molecules or miRNA molecules. Most particularly, they are miRNA molecules. A difference between shRNA molecules and miRNA molecules is that miRNA molecules are processed by Drosha, while conventional shRNA molecules are not (which has been associated with toxicity, Grimm et al., Nature 441:537-541 (2006)).

According to specific embodiments, the miRNA molecules can be provided as one miRNA scaffold under control of one promoter. If the scaffold chosen normally harbors one miRNA, the scaffold can be repeated or combined with other scaffolds to obtain the expression of multiple RNA interference molecules. However, when repeating or combining with further scaffolds, it is typically envisaged that all of the multiplexed RNA interference molecules will be under control of one promoter (i.e., the promoter is not repeated when the single scaffold is repeated).

Particularly suited scaffold sequences for miRNA multiplexing are a miR-30 scaffold sequence, a miR-155 scaffold sequence, and a miR-196a2 scaffold sequence. However, according to particular embodiments, no miR-30 or miR-155 sequences are used.

Typically, at least one of the miRNA molecules comprises a miR-196 scaffold sequence, preferably a miR-196a2 scaffold sequence. According to specific embodiments, all of the at least two miRNA molecules comprise a miR-scaffold sequence, preferably a miR-196a2 scaffold sequence. The same can be said for miR-30 and miR-155 scaffold sequences. Examples of such suitable scaffolds are listed in U.S. Pat. No. 8,841,267 (particularly claim 1 therein), incorporated herein by reference. The single scaffold is commercially available as the SMARTvector™ micro-RNA adapted scaffold (Horizon Discovery, Lafayette, Colo., USA).

Further suitable scaffold sequences include miR-26b (hsa-mir-26b), miR-204 (hsa-mir-204), and miR-126 (hsa-mir-126), hsa-let-7f, hsa-let-7g, hsa-let-7a, hsa-let-7b, hsa-let-7c, hsa-mir-29a, hsa-mir-140-3p, hsa-let-7i, hsa-let-7e, hsa-mir-7-1, hsa-mir-7-2, hsa-mir-7-3, hsa-mir-26a, hsa-mir-26a, hsa-mir-340, hsa-mir-101, hsa-mir-29c, hsa-mir-191, hsa-mir-222, hsa-mir-34c-5p, hsa-mir-21, hsa-mir-378, hsa-mir-100, hsa-mir-192, hsa-mir-30d, hsa-mir-16, hsa-mir-432, hsa-mir-744, hsa-mir-29b, hsa-mir-130a, or hsa-mir-15a.

According to alternative, but not exclusive embodiments, rather than using a particular miR scaffold that is repeated, resulting in an artificially repeated scaffold, authentic polycistronic miRNA clusters or parts thereof can be used, where the endogenous miRNA is replaced by shRNA of interest. Particularly suitable miR scaffold clusters to this end are miR-106a^{˜}363, miR-17^{˜}92, miR-106b^{˜}25, and miR-23a^{˜}27a^{˜}24-2 cluster; most particularly envisaged is the miR-106a^{˜}363 cluster and fragments thereof. Of note, to save vector payload, it is also specifically envisaged to use part of such natural clusters and not all of the sequences (this is particularly useful as not all miRNAs are equally interspaced, and not all linker sequences may be needed). Other considerations can be taken into account, e.g. taking the miRNAs that are most efficiently processed in a cell. For instance, the miR-17^{˜}92 cluster consists of (in order) miR-17, miR-18a, miR-19a, miR-20a, miR-19b-1 and miR-92-1 (also miR-92a1), particularly useful fragments thereof are the scaffold sequence from miR-19a to miR-92-1 (i.e. 4 of the 6 miRNAs) or from miR-19a to miR-19b-1 (3 of the 6 miRNAs). Likewise, the 106a^{˜}363 cluster consists of (in order) miR-106a, miR-18b, miR-20b, miR-19b-2, miR-92-2 (also miR-92a2) and miR-363. Particularly useful fragments thereof are the scaffold sequence from miR-20b to miR-363 (i.e. 4 of the 6 miRNAs) or from miR-19b-2 to miR-363 (i.e. 3 of the 6 miRNAs). Both the natural linker sequences can be used, as well as fragments thereof or artificial linkers (again to reduce payload of the vectors).

It is envisaged that a combination of these strategies can be used, e.g. both the miR-106a^{˜}363 cluster and a miR-196a2 sequence can be combined in a novel scaffold.

The cells disclosed herein contain multiplexed RNA interference molecules. These can be directed against one or more targets which need to be downregulated (either targets within the cell, or outside of the cell if the shRNA is secreted). Each RNA interference molecule can target a different molecule, they can target the same molecule, or a combination thereof (i.e. more than one RNA molecule directed against one target, while only one RNA interference molecule is directed against a different target). When the RNA interference molecules are directed against the same target, they can target the same region, or they can target a different region. In other words, the RNA interference molecules can be identical or not when directed against the same target. Examples of such combinations of RNA interference molecules are shown in Example 9.

Thus, according to particular embodiments, at least two of the multiplexed RNA interference molecules are directed against the same target. According to further specific embodiments, at least two of the multiplexed RNA interference molecules are identical.

According to alternative embodiments, all of the at least two multiplexed RNA interference molecules are different. According to further specific embodiments, all of the at least two multiplexed RNA interference molecules are directed against different targets.

Any suitable molecule present in the engineered cell can be targeted by the instant RNA interference molecules. Typical examples of envisaged targets are: a MHC class I gene, a MHC class II gene, a MHC coreceptor gene (e.g. HLA-F, HLA-G), a TCR chain, a CD3 chain, NKBBiL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCP5, DGKA, DGKZ, B2M, MICA, MICB, ULBP1, ULBP2, ULBP3, ULBP4, ULBP5, ULBP6, 2B4, AZAR, BAX, BLIMP1, C160 (POLR3A), CBL-B, CCR6, CD7, CD95, CD123, DGK [DGKA, DGKB, DGKD, DGKE, DKGG, DGKH, DGKI, DGKK, DGKQ, DGKZ], DNMT3A, DR4, DR5, EGR2, FABP4, FABP5, FASN, GMCSF, HPK1, IL-10R [IL10RA, IL10RB], IL2, LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, SHIP1, SOAT1, SOCS1, T-BET, TET2, TGFBR1, TGFBR2, TGFBR3, TIGIT, TIM3, TOX, and ZFP36L2.

Particularly suitable constructs have been identified which are miRNA-based. Accordingly, provided are engineered cells comprising a polynucleotide comprising a multiplexed microRNA-based shRNA encoding region, wherein said multiplexed microRNA-based shRNA encoding region comprises sequences that encode:

two or more artificial miRNA-based shRNA nucleotide sequences, wherein each artificial miRNA-based shRNA nucleotide sequence comprises
a miRNA scaffold sequence,
an active or mature sequence, and
a passenger or star sequence, wherein within each artificial miRNA-based shRNA nucleotide sequence, the active sequence is at least 80% complementary to the passenger sequence.

Both the active sequence and the passenger sequence of each of the artificial miRNA-based shRNA nucleotide sequences are typically between 18 and 40 nucleotides long, more particularly between 18 and 30 nucleotides, most particularly between 19 and 25 nucleotides long.

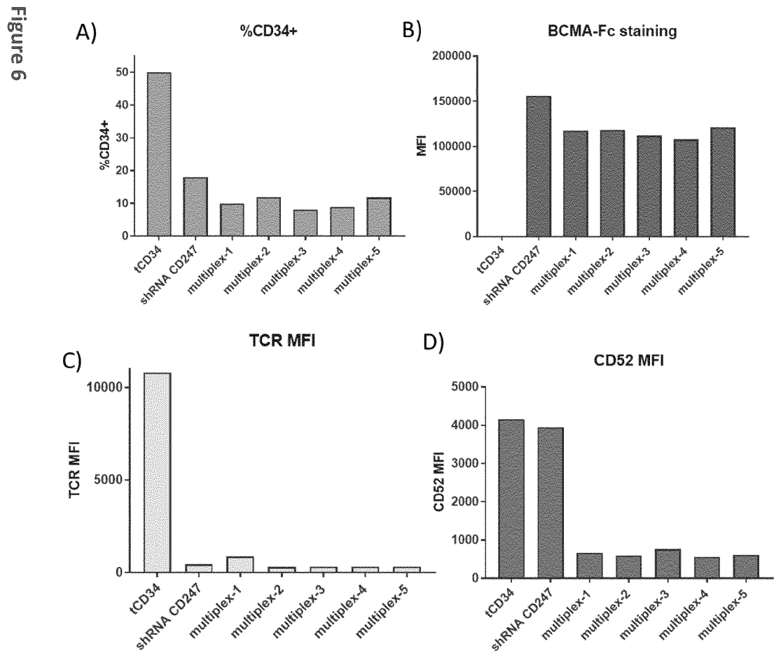

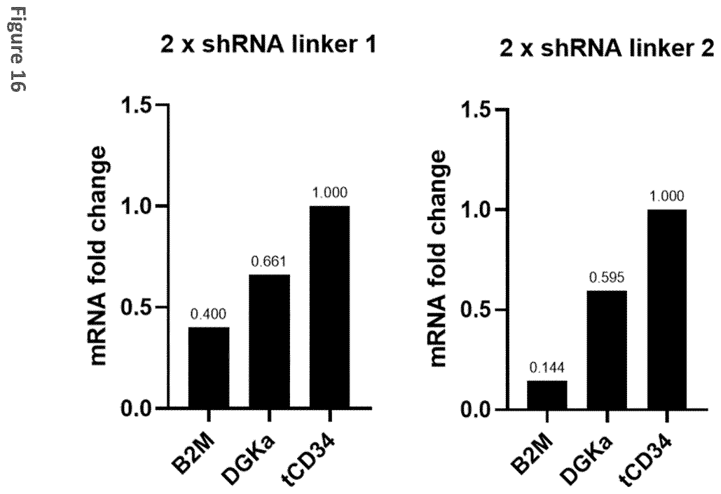

Typically, these microRNA scaffold sequences are separated by linkers, and linker sequences can e.g. be between 30 and 60 nucleotides long, although shorter stretches also work. In fact, it was surprisingly found that length of linker plays no vital role and can be very short (less than 10 nucleotides) or even be absent without interfering with shRNA function. This is shown e.g. in FIGS. 6 and 16.

Artificial sequences can e.g. be naturally occurring scaffolds (e.g. a miR cluster or fragment thereof, such as the miR-106a^{˜}363 cluster) wherein the endogenous miR sequences have been replaced by shRNA sequences engineered against a particular target, can be repeats of a single miR scaffold (such as e.g. the miR-196a2 scaffold) wherein the endogenous miR sequences have been replaced by shRNA sequences engineered against a particular target, can be artificial miR-like sequences, or a combination thereof.

This engineered cell typically further comprises a nucleic acid molecule encoding a protein of interest, such as a chimeric antigen receptor or a TCR, and can be an engineered immune cell, as described above.

The co-expression of the multiplexed RNA interference molecules results in the suppression of at least one gene, but typically a plurality of genes, within the engineered cells. This can contribute to greater therapeutic efficacy.

The engineered cells described herein are also provided for use as a medicament. According to specific embodiments, the engineered cells are provided for use in the treatment of cancer. Exemplary types of cancer that can be treated include, but not limited to, adenocarcinoma, adrenocortical carcinoma, anal cancer, astrocytoma, bladder cancer, bone cancer, brain cancer, breast cancer, cervical cancer, colorectal cancer, endometrial cancer, esophageal cancer, Ewing sarcoma, eye cancer, Fallopian tube cancer, gastric cancer, glioblastoma, head and neck cancer, Kaposi sarcoma, kidney cancer, leukemia, liver cancer, lung cancer, lymphoma, melanoma, mesothelioma, myelodysplastic syndrome, multiple myeloma, neuroblastoma, osteosarcoma, ovarian cancer, pancreatic cancer, parathyroid cancer, penile cancer, peritoneal cancer, pharyngeal cancer, prostate cancer, renal cell carcinoma, retinoblastoma, rhabdomyosarcoma, sarcoma, skin cancer, small intestine cancer, stomach cancer, testicular cancer, thyroid cancer, urethral cancer, uterine cancer, vaginal cancer, and Wilms tumor.

According to particular embodiments, the cells can be provided for treatment of liquid or blood cancers. Examples of such cancers include e.g. leukemia (including a.o. acute myelogenous leukemia (AML), acute lymphocytic leukemia (ALL), chronic myelogenous leukemia (CML), and chronic lymphocytic leukemia (CLL)), lymphoma (including a.o. Hodgkin's lymphoma and non-Hodgkin's lymphoma such as B-cell lymphoma (e.g. DLBCL), T cell lymphoma, Burkitt's lymphoma, follicular lymphoma, mantle cell lymphoma, and small lymphocytic lymphoma), multiple myeloma or myelodysplastic syndrome (MDS).

This is equivalent as saying that methods of treating cancer are provided, comprising administering to a subject in need thereof a suitable dose of engineered cells as described herein (i.e. engineered cells comprising an exogenous nucleic acid molecule encoding at least two multiplexed RNA interference molecules, and optionally comprising a further nucleic acid molecule encoding a protein of interest), thereby improving at least one symptom associated with the cancer. Cancers envisaged for treatment include, but are not limited to, adenocarcinoma, adrenocortical carcinoma, anal cancer, astrocytoma, bladder cancer, bone cancer, brain cancer, breast cancer, cervical cancer, colorectal cancer, endometrial cancer, esophageal cancer, Ewing sarcoma, eye cancer, Fallopian tube cancer, gastric cancer, glioblastoma, head and neck cancer, Kaposi sarcoma, kidney cancer, leukemia, liver cancer, lung cancer, lymphoma, melanoma, mesothelioma, myelodysplastic syndrome, multiple myeloma, neuroblastoma, osteosarcoma, ovarian cancer, pancreatic cancer, parathyroid cancer, penile cancer, peritoneal cancer, pharyngeal cancer, prostate cancer, renal cell carcinoma, retinoblastoma, rhabdomyosarcoma, sarcoma, skin cancer, small intestine cancer, stomach cancer, testicular cancer, thyroid cancer, urethral cancer, uterine cancer, vaginal cancer, and Wilms tumor. According to further particular embodiments, methods of treating blood cancer are provided, comprising administering to a subject in need thereof a suitable dose of engineered cells as described herein thereby improving at least one symptom of the cancer.

According to alternative embodiments, the cells can be provided for use in the treatment of autoimmune disease. Exemplary types of autoimmune diseases that can be treated include, but are not limited to, rheumatoid arthritis (RA), systemic lupus erythematosus (SLE), inflammatory bowel disease (IBD), multiple sclerosis (MS), Type 1 diabetes mellitus, amyotrophic lateral sclerosis (ALS or Lou Gehrig's disease), spinal muscular atrophy (SMA), Crohn's disease, Guillain-Barre syndrome, chronic inflammatory demyelinating polyneuropathy, psoriasis, psoriatic arthritis, Addison's disease, ankylosing spondylitis, Behcet's disease, coeliac disease, Coxsackie myocarditis, endometriosis, fibromyalgia, Graves' disease, Hashimoto's thyroiditis, Kawasaki disease, Meniere's disease, myasthenia gravis, sarcoidosis, scleroderma, Sjögren's syndrome, thrombocytopenic purpura (TTP), ulcerative colitis, vasculitis and vitiligo.

This is equivalent as saying that methods of treating autoimmune disease are provided, comprising administering to a subject in need thereof a suitable dose of engineered cells as described herein, thereby improving at least one symptom associated with the autoimmune disease. Exemplary autoimmune diseases that can be treated are listed above.

According to yet further embodiments, the cells can be provided for use in the treatment of infectious disease. “Infectious disease” is used herein to refer to any type of disease caused by the presence of an external organism (pathogen) in or on the subject or organism with the disease. Infections are usually considered to be caused by microorganisms or microparasites like viruses, prions, bacteria, and viroids, though larger organisms like macroparasites and fungi can also infect. The organisms that can cause infection are herein referred to as “pathogens” (in case they cause disease) and “parasites” (in case they benefit at the expense of the host organism, thereby reducing biological fitness of the host organism, even without overt disease being present) and include, but are not limited to, viruses, bacteria, fungi, protists (e.g. Plasmodium, Phytophthora) and protozoa (e.g. Plasmodium, Entamoeba, Giardia, Toxoplasma, Cryptosporidium, Trichomonas, Leishmania, Trypanosoma) (microparasites) and macroparasites such as worms (e.g. nematodes like ascarids, filarias, hookworms, pinworms and whipworms or flatworms like tapeworms and flukes), but also ectoparasites such as ticks and mites. Parasitoids, i.e. parasitic organisms that sterilize or kill the host organism, are envisaged within the term parasites. According to particular embodiments, the infectious disease is caused by a microbial or viral organism.

“Microbial organism,” as used herein, may refer to bacteria, such as gram-positive bacteria (eg, Staphylococcus sp., Enterococcus sp., Bacillus sp.), Gram-negative bacteria. (for example, Escherichia sp., Yersinia sp.), spirochetes (for example, Treponema sp, such as Treponema pallidum, Leptospira sp., Borrelia sp., such as Borrelia burgdorferi), mollicutes (i.e. bacteria without cell wall, such as Mycoplasma sp.), acid-resistant bacteria (for example, Mycobacterium sp., such as Mycobacterium tuberculosum, Nocardia sp.). “Microbacterial organisms” also encompass fungi (such as yeasts and molds, for example, Candida sp., Aspergillus sp., Coccidioides sp., Cryptococcus sp., Histoplasma sp., Pneumocystis sp. Or Trichophyton sp.), Protozoa (for example, Plasmodium sp., Entamoeba sp., Giardia sp., Toxoplasma sp., Cryptosporidium sp., Trichomonas sp., Leishmania sp., Trypanosoma sp.) and archaea. Further examples of microbial organisms causing infectious disease that can be treated with the instant methods include, but are not limited to, Staphylococcus aureus (including methicillin-resistant S. aureus (MRSA)), Enterococcus sp. (including vancomycin-resistant enterococci (VRE), the nosocomial pathogen Enterococcus faecalis), food pathogens such as Bacillus subtilis, B. cereus, Listeria monocytogenes, Salmonella sp., and Legionella pneumophilia.

“Viral organism” or “virus”, which are used as equivalents herein, are small infectious agents that can replicate only inside the living cells of organisms. They include dsDNA viruses (e.g. Adenoviruses, Herpesviruses, Poxviruses), ssDNA viruses (e.g. Parvoviruses), dsRNA viruses (e.g. Reoviruses), (+)ssRNA viruses (e.g. Picornaviruses, Togaviruses, Coronaviruses), (−)ssRNA viruses (e.g. Orthomyxoviruses, Rhabdoviruses), ssRNA-RT (reverse transcribing) viruses, i.e. viruses with (+)sense RNA with DNA intermediate in life-cycle (e.g. Retroviruses), and dsDNA-RT viruses (e.g. Hepadnaviruses). Examples of viruses that can also infect human subjects include, but are not limited to, an adenovirus, an astrovirus, a hepadnavirus (e.g. hepatitis B virus), a herpesvirus (e.g. herpes simplex virus type I, the herpes simplex virus type 2, a Human cytomegalovirus, an Epstein-Barr virus, a varicella zoster virus, a roseolovirus), a papovavirus (e.g. the virus of human papilloma and a human polyoma virus), a poxvirus (e.g. a variola virus, a vaccinia virus, a smallpox virus), an arenavirus, a buniavirus, a calcivirus, a coronavirus (e.g. SARS coronavirus, MERS coronavirus, SARS-CoV-2 coronavirus (etiologic agent of COVID-19)), a filovirus (e.g. Ebola virus, Marburg virus), a flavivirus (e.g. yellow fever virus, a western Nile virus, a dengue fever virus, a hepatitis C virus, a tick-borne encephalitis virus, a Japanese encephalitis virus, an encephalitis virus), an orthomyxovirus (e.g. type A influenza virus, type B influenza virus and type C influenza virus), a paramyxovirus (e.g. a parainfluenza virus, a rubulavirus (mumps), a morbilivirus (measles), a pneumovirus, such as a human respiratory syncytial virus), a picornavirus (e.g. a poliovirus, a rhinovirus, a coxackie A virus, a coxackie B virus, a hepatitis A virus, an ecovirus and an enterovirus), a reovirus, a retrovirus (e.g. a lentivirus, such as a human immunodeficiency virus and a human T lymphotrophic virus (HTLV)), a rhabdovirus (e.g. rabies virus) or a togavirus (e.g. rubella virus). According to particular embodiments, the infectious disease to be treated is not HIV. According to alternative embodiments, the infectious disease to be treated is not a disease caused by a retrovirus. According to alternative embodiments, the infectious disease to be treated is not a viral disease.

This is equivalent as saying that methods of treating infectious disease are provided, comprising administering to a subject in need thereof a suitable dose of engineered cells as described herein (i.e. engineered cells comprising an exogenous nucleic acid molecule encoding two or more multiplexed RNA interference molecules, and optionally comprising a further nucleic acid molecule encoding a protein of interest), thereby improving at least one symptom. Particularly envisaged microbial or viral infectious diseases are those caused by the pathogens listed above.

These cells that are provided for use as a medicament can be provided for use in allogeneic therapies. I.e., they are provided for use in treatments where allogeneic ACT is considered a therapeutic option (wherein cells from another subject are provided to a subject in need thereof). According to specific embodiments, in allogeneic therapies, at least one of the RNA interference molecules will be directed against the TCR (most particularly, against a subunit of the TCR complex). According to alternative embodiments, these cells are provided for use in autologous therapies, particularly autologies ACT therapies (i.e., with cells obtained from the patient).

It is to be understood that although particular embodiments, specific configurations as well as materials and/or molecules, have been discussed herein for cells and methods according to present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1. Evaluation of miRNA Scaffold Length in Downregulating TCR

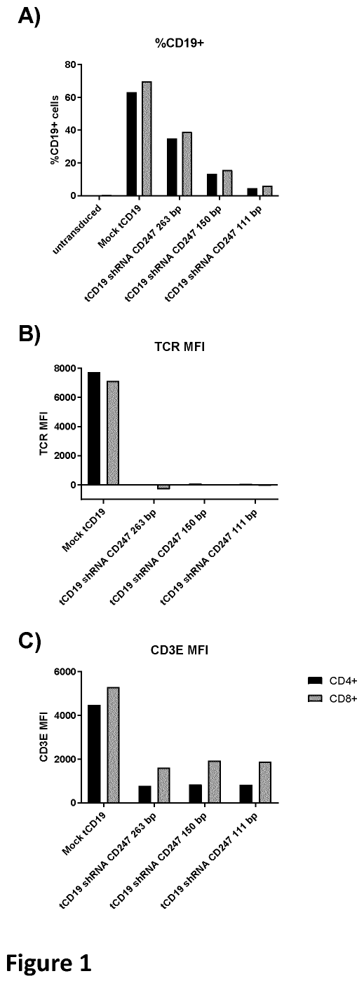

Successful multiplexing of different shRNAs in the same viral expression vector desires the miRNA-based scaffold to be as small as possible. This would allow for the combination of multiple shRNAs, without significantly affecting the overall size of the vector. In order to assess whether shortened miRNA-based shRNA scaffolds were still efficient in target knockdown, we expressed a previously identified CD247 (a TCR subunit) targeting shRNA from a miRNA-196a2 scaffold (SMARTvector™ micro-RNA adapted scaffold (Horizon Discovery, Lafayette, Colo., USA)), where the new constructs differed in their miRNA scaffold length. The original construct has 263 nucleic acids, two shortened constructs used 150 or 111 nucleic acids respectively. All viral vectors were able to transduce primary T cells, albeit to different degrees (FIG. 1), as indicated by a truncated CD19 marker. However, the degree of TCR or CD3E protein knockdown was comparable for all three constructs, indicating that also a minimal miRNA-based shRNA scaffold is still able to reduce TCR/CD3 complex expression to a similar degree as the longer miRNA scaffold.

### Example 2. Screening of Different CD52 Targeting shRNAs

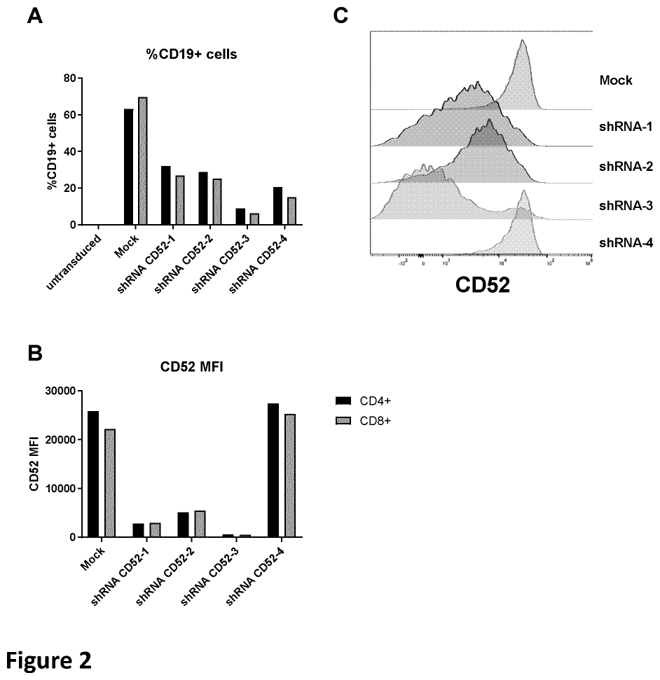

To be able to compare the efficiency of different CD52 targeting shRNAs, an identical backbone construct to the one previously employed with CD247 was used, where only the targeting sequence of the shRNA was exchanged, placing a CD52-targeting instead of the CD247-targeting sequence. A retroviral vector was used to deliver the shRNAs into primary T cells, which could be tracked by a truncated CD19 (tCD19) marker. Different CD52-targeting shRNAs were cloned into the miRNA scaffold of the retroviral vector. The degree of CD52 knockdown was assessed on day 8 of the cell culture. All constructs were able to transduce primary T cells, as measured by CD19-expression (FIG. 2). However, the degree of CD52 knockdown was different for the 4 shRNAs tested. Considering knocking down CD52 expression, the shRNA-3 was most efficient, followed by shRNA-1 and shRNA-2. In contrast, shRNA-4 showed no decrease in CD52 expression (FIG. 2).

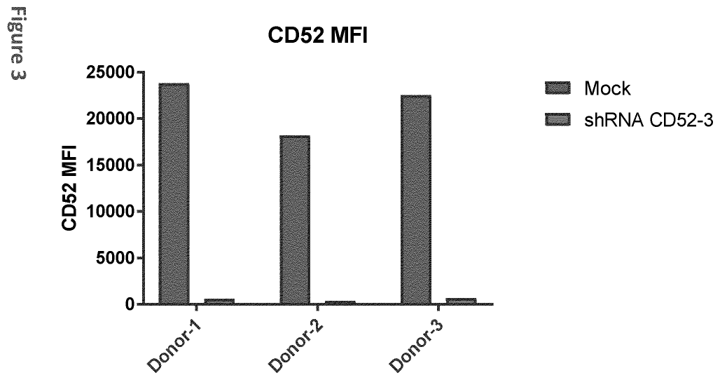

In order to assess whether knockdown efficiency of this shRNA would vary between different donors, T cells from 3 different donors were transduced with a Mock construct or a construct expression the CD52 shRNA-3 (FIG. 3). In all three donors the shRNA-3 showed a significant and consistent knockdown of CD52 (FIG. 3), indicating that the identified shRNA results in consistent and donor-independent knockdown of CD52.

### Example 3. Screening of gRNAs for CRISPR/Cas9 Mediated CD52 Knockout

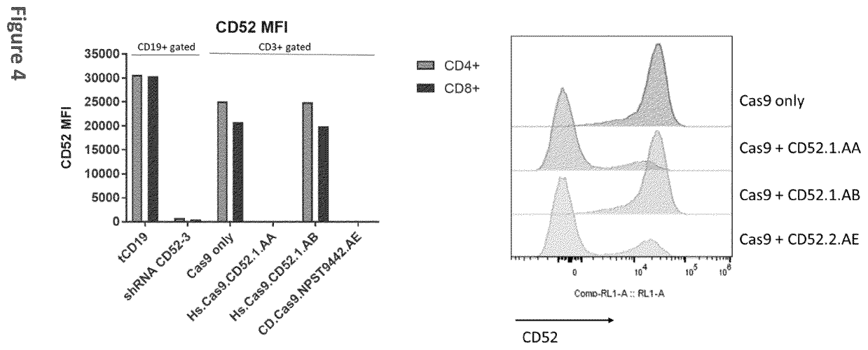

In order to generate a positive control for CD52 and/or TCR/CD3 complex expression inhibition, the respective knockout T cells were generated using CRISPR/Cas9 technology. Different guide RNAs (gRNAs) were designed and assessed to identify the ones that would efficiently generate CD52-deficient T cells (FIG. 4). Two out of three gRNAs were able to generate CD52 knockout cells (gRNA-1, indicated as CD52.1.AA in FIG. 4, and gRNA-3, indicated as CD52.2.AE in FIG. 4), with the frequency of CD52-deficient cells being slightly higher with CD52 gRNA-1.

### Example 4. Effect of miRNA Spacers on Target Knockdown

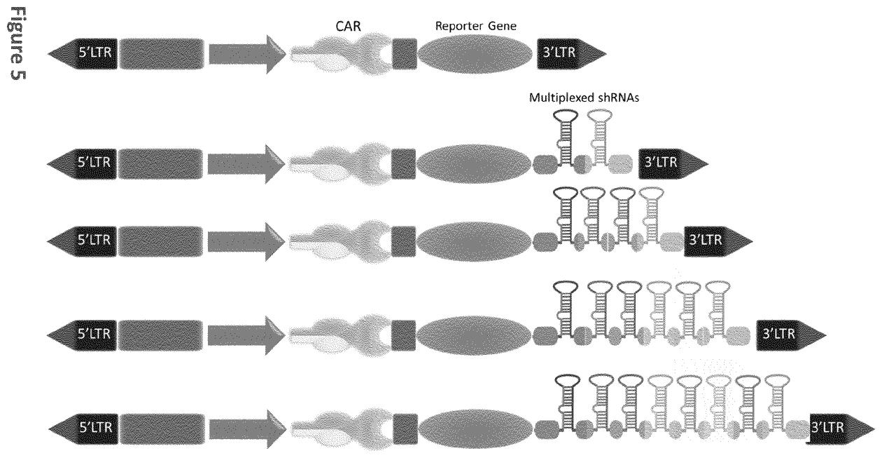

Efficient processing of the miRNA from the transcribed RNA, by the DROSHA complex, is pivotal for efficient target knockdown. Our previous data showed that miRNA based shRNAs could efficiently be co-expressed with a CAR-encoding vector and processed by the miRNA machinery from the vector. It would further be desirable to generate a CAR expression vector, capable of co-expressing multiple miRNA based shRNAs (e.g. 2, 4, 6, 8 . . . ) from the same vector (FIG. 5). However, previous studies showed that co-expression of multiple miRNA-based shRNAs leads to loss of shRNA activity. Thus, for knocking down multiple targets from a single expression vector, efficient miRNA processing is important.

In order to optimize activity of two co-expressed shRNAs, we hypothesized that not only the size, but also the sequence of the linker between two miRNA-based shRNAs, as well as the miRNA scaffold would affect shRNA activity. In order to optimize the shRNA processing, we assessed the impact of different shRNA linkers on the knockdown of two target genes, CD247 (CD3ζ) and CD52. Five different spacers based on spacer sequences derived from the natural occurring human miR-17-92 cluster were designed. The five different spacers were cloned between a CD247 and the CD52 shRNA in the context of a BCMA CAR. T cells were transduced with the different constructs, using a tCD34 (Mock) and BCMA-CD247 shRNA vector as control; results are shown in FIG. 6. Multiplex 1 contained no spacer between the 111 bp CD247 shRNA and the 111 bp CD52 shRNA. Multiplex 2 contained the 43bp naturally occurring spacer between miR-17 and miR-18a in the miR-17-92 cluster. Multiplex 3 contained a 92bp spacer, corresponding to the spacer region between miR-19a and miR-20a in the miR-17-92 cluster. Multiplex 4 contained a 56bp spacer, corresponding to the spacer region between miR-20a and miR-19b1 in the miR-17-92 cluster. Multiplex 5 contained a random TA rich spacer region of 29bp. All constructs with the shRNA showed a low, but comparable transduction efficiency at harvest. Also, expression of the BCMA CAR was only slightly affected by the expression of multiple shRNAs (FIG. 6). Assessment of CD52 and TCR knockdown showed that all constructs were able to decrease TCR and CD52 expression at comparable levels. Only the first multiplex construct, lacking any spacer between the two hairpins, showed a slightly lower knockdown activity for TCR (but not for CD52) compared to the other constructs, but it still worked very well in reducing expression (FIG. 6).

### Example 5. Comparing Multiplexed and Single shRNAs

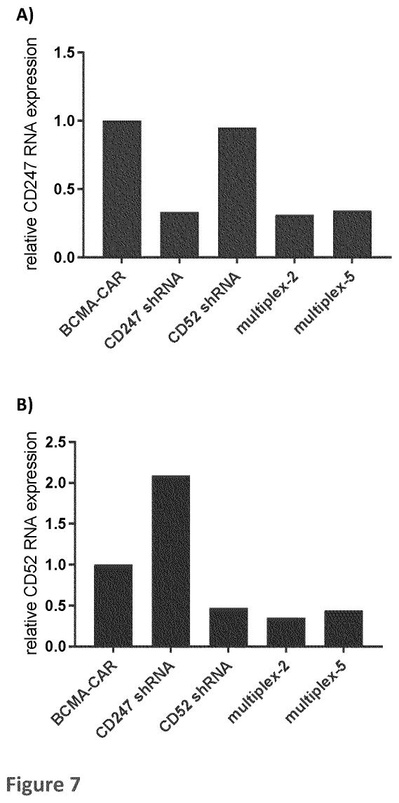

Next, we aimed to directly compare the effect of multiplexing two shRNAs to the effect of expression of single shRNAs for CD247 and CD52. RNA expression analysis showed that the multiplexed shRNA constructs were as efficient in downregulating CD52 or CD247 as the respective single shRNAs (FIG. 7).

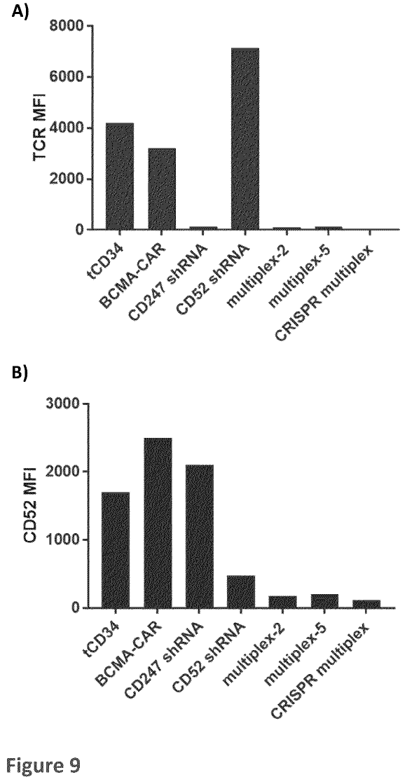

As another control, we used the CRISPR/Cas9 system concurrently targeting CD52 and CD247 using two different gRNAs. At harvest, cells containing CD247 shRNA or gRNA were depleted for TCR-positive cells before further analysis. The TCR and CD52 expression was assessed by flow cytometry, in order to compare the protein expression of cells transduced with a single or multiplexed shRNAs (FIGS. 8 and 9). The single CD247 shRNA was able to reduce expression of TCR (FIGS. 8 and 9). The reduction of TCR surface expression was comparable to a CRISPR/Cas9 mediated knockout of CD247. Similarly, a single CD52 shRNA was able to reduce CD52 expression (FIGS. 8 and 9). The two multiplexed shRNA constructs with different linkers (see Example 4), both showed the same degree of TCR knockdown as the single shRNA. Similarly, the CD52 expression was reduced to the same extend by a single or the multiplexed shRNA constructs (FIGS. 8 and 9).

### Example 6. CAR Expression and Cell Potency

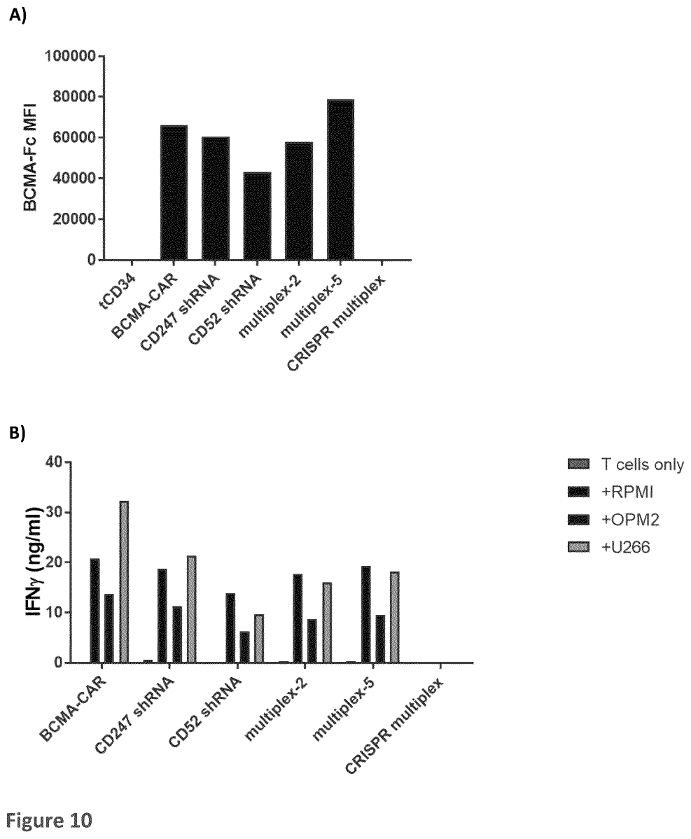

In order to assess the influence of the co-expression of one or multiple shRNAs on the CAR expression and functionality, BCMA-CAR expression was assessed by flow cytometry. Cells were stained with BCMA-Fc fusion protein, followed by a staining with a secondary PE-conjugated antibody. As shown in FIG. 10, the expression of the CAR was similar between all groups, which shows that these multiplexed shRNAs did not affect the levels of CAR expression. Furthermore, we assessed functional activity of BCMA-CAR expressing cells against the BCMA-positive cancer cell lines RPMI-8226, OPM-2 and U226 (FIG. 10). To this end, T cells were co-cultured for 24 h with cancer cells, before assessing IFNγ levels in the supernatant. T cells alone did not produce any IFNγhowever, co-culture with BCMA-expressing cancer cells resulted in comparable IFNγ production by all groups of T cells. Thus, co-expression of one or multiple shRNAs does not influence the expression of the CAR or the functional activity of the CAR-T cells against cancer cell lines.

### Example 7. Functional Response of CAR T Cells to Mitogenic Stimuli

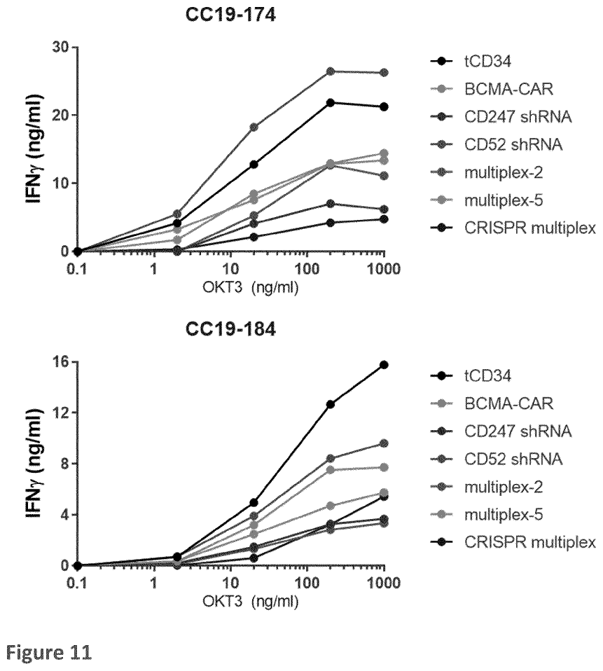

Next, the reactivity of the CAR-T cells towards a mitogenic TCR stimulation was assessed. To this end, T cells were stimulated with increasing concentrations of an anti-CD3 antibody (clone OKT3) and IFNγ production was measured after 24 h. Mock-transduced cells produced high levels of IFNγ after OKT3 activation. Similarly, co-expression of a BCMA-CAR alone, or in combination with a CD52 shRNA did not reduce the capacity of T cells to respond to TCR activation stimuli. However, co-expression of a CD247 shRNA single, or multiplexed, did reduce functional responses of TCR significantly to the levels of CIRSPR/Cas9 CD247 genome edited control cells (FIG. 11). Thus, multiplexed shRNAs are as efficient in inhibiting TCR function as the single shRNA control and genome edited T cells.

### Example 8. Functional Inhibition of CD52

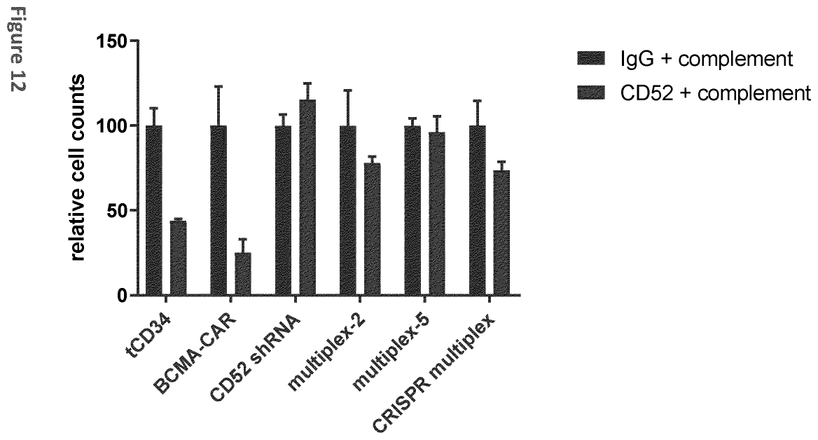

In a next step, we aimed to assess how expression of a single or multiplexed CD52 shRNA would affect the complement dependent killing of T cells in the presence of an anti-CD52 antibody. To this end, T cells were cultured with complement in the presence of an anti-CD52 antibody (alemtuzumab) or a control IgG antibody. After 4 h, cell numbers were determined. Mock and BCMA-CAR transduced T cells were efficiently targeted by the complement system in the presence of alemtuzumab (FIG. 12). However, both, single and multiplexed CD52 shRNA was able to prevent CD52-mediated killing.

### Example 9. Multiplexing of More than 2 Targets

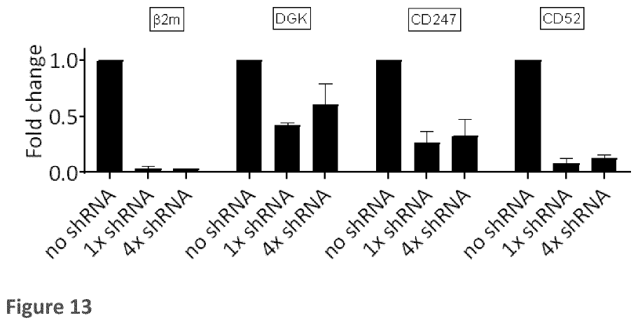

Next the feasibility of multiplexing four shRNAs was assessed. To assess this, Jurkat cells were transduced with either single or multiplexed shRNAs targeting β2m, DGK, CD247 (CD3ζ) and CD52, along with a second generation CD19 CAR and a selection marker using a lentiviral backbone and making use of a repeated miR-196a2 scaffold. Single step enrichment was performed using marker-specific magnetic beads on day 7 after transduction. Cells were analyzed for shRNA targets expression by qRT-PCR. The shRNA-mediated downregulation of the transcriptional expression of the four targets was equivalent between the multiplexes and the respective single shRNA (FIG. 13).

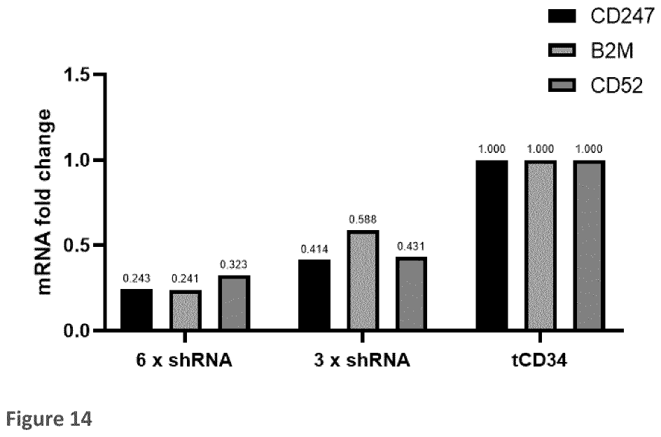

Next to Jurkat cells, primary T cells were transduced with retroviral vectors encoding a second generation CD19 CAR containing either 3×sRNAs or 6×sRNAs targeting CD247, β2m and CD52 introduced in the miR-106a-363 cluster (FIG. 14). Briefly, primary T cells from a healthy donor were transduced with retroviral vectors encoding a second generation CD19-directed CAR, a truncated CD34 selection marker along with 3 shRNAs targeting CD247, B2M and CD52, introduced in the last three miRs of the 106a-363miRNA cluster (miR-19b2, miR-92a2 and miR-363), or 6 shRNAs targeting the same three genes in the 6 miR scaffolds of the cluster (in this case the two shRNAs targeting CD247 were different). Concisely, shRNAs expressed as a 6-plex, 3-plex or no shRNA (tCD34) as control. Two days after transduction, cells were enriched using CD34-specific magnetic beads, and further amplified in IL-2 (100 IU/mL) for 6 days. mRNA expression of CD247, B2M and CD52 was assessed by qRT-PCR using cyclophilin as house-keeping gene.

Multiplexed shRNAs yielded efficient RNA knock-down levels for all targeted genes. Incorporation of six multiplexed shRNAs (two shRNAs against each protein target) resulted in higher RNA knock-down levels compared to three multiplexed shRNAs (one shRNA against each protein target) (FIG. 14).

### Example 10. Multiplex Knockdown of Targets in iPSC Cells

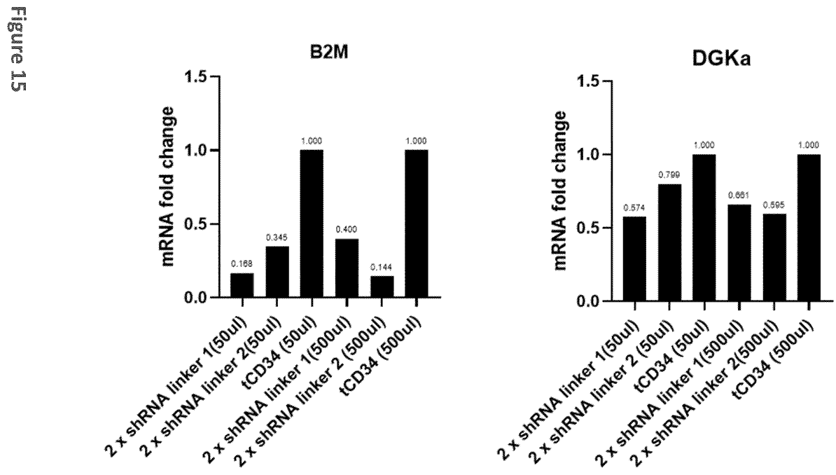

To explore knockdown of multiplexed RNA interference molecules in other immune cells, multiplexing was next assessed in iPSCs. Two shRNAs (against β2m and DGKa) separated by either a long (41bp) or minimal (6bp) linker were expressed in the human iPSC cell line SCiPS-R1. Transduction was performed with either 50 μl or 500 μl of viral supernatant. Multiplexed shRNAs yielded efficient RNA knock-down levels independently of the linker size or the volume of the viral supernatant used when compared to cells transduced with no shRNA (FIG. 15, FIG. 16).

1. An engineered cell comprising:
a first exogenous nucleic acid molecule encoding a protein of interest, and
a second nucleic acid molecule encoding at least two multiplexed RNA interference molecules.

2. The engineered cell of claim 1, which is an engineered immune cell.

3. The engineered immune cell of claim 1 or 2, wherein the immune cell is selected from a T cell, a NK cell, a NKT cell, a stem cell, a progenitor cell, and an iPSC cell.

4. The engineered cell of any one of claims 1 to 3, wherein the protein of interest is a receptor, particularly a chimeric antigen receptor or a TCR.

5. The engineered cell of any one of claims 1 to 4, wherein the first and second nucleic acid molecule are present in one vector, such as a eukaryotic expression plasmid, a mini-circle DNA, or a viral vector (e.g. derived from a lentivirus, a retrovirus, an adenovirus, an adeno-associated virus, and a Sendai virus).

6. The engineered cell of any one of claims 1 to 5, wherein the at least two multiplexed RNA interference molecules are under control of one promoter.

7. The engineered cell of claim 6, wherein the promoter is not a U6 promoter.

8. The engineered cell of claim 6, wherein the promoter is a Pol II promoter selected from the group consisting of a CMV promoter, an EF1α promoter (core or full length), a PGK promoter, a CAG promoter, a UbC promoter, a SFFV promoter, a RSV promoter, a IL-2 promoter, a MSCV LTR, a SV40 promoter, GALV LTR and a tRNA promoter.

9. The engineered cell of any one of claims 1 to 8, wherein the at least two multiplexed RNA interference molecules are miRNA molecules.

10. The engineered cell of claim 9, wherein the miRNA molecules are one miRNA scaffold under control of one promoter.

11. The engineered cell of claim 9, wherein at least one of the miRNA molecules comprises a miR-196a2 scaffold sequence or a scaffold sequence from the miR-106a^{˜}363 cluster.

12. The engineered cell of claim 11, wherein all of the at least two miRNA molecules comprise a miR-scaffold sequence, preferably a miR-196a2 scaffold sequence or a scaffold sequence from the miR-106a^{˜}363 cluster.

13. The engineered cell of any one of claims 1 to 12, wherein at least two of the multiplexed RNA interference molecules are directed against the same target.

14. The engineered cell of claim 13, wherein at least two of the multiplexed RNA interference molecules are identical.

15. The engineered cell of any one of claims 1 to 12, wherein all of the at least two multiplexed RNA interference molecules are different.

16. The engineered cell of claim 15, wherein all of the at least two multiplexed RNA interference molecules are directed against different targets.

17. The engineered cell of any one of claims 1 to 16, wherein the molecule targeted by the at least two multiplexed RNA interference molecules is selected from: a MHC class I gene, a MHC class II gene, a MHC coreceptor gene (e.g. HLA-F, HLA-G), a TCR chain, NKBBiL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCPS, DGKA, DGKZ, B2M, MICA, MICB, ULBP1, ULBP2, ULBP3, ULBP4, ULBPS, ULBP6, 2B4, A2AR, BAX, BLIMP1, C160 (POLR3A), CBL-B, CCR6, CD7, CD95, CD123, DGK [DGKA, DGKB, DGKD, DGKE, DKGG, DGKH, DGKI, DGKK, DGKQ, DGKZ], DNMT3A, DR4, DRS, EGR2, FABP4, FABPS, FASN, GMCSF, HPK1, IL-10R [IL10RA, IL10RB], IL2, LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, SHIP1, SOAT1, SOCS1, T-BET, TET2, TGFBR1, TGFBR2, TGFBR3, TIGIT, TIM3, TOX, and ZFP36L2.

18. The engineered cell of any one of claims 1 to 17 for use as a medicament.

19. The engineered cell of any one of claims 1 to 18 for use in the treatment of cancer.

20. A method of treating cancer, comprising administering to a subject in need thereof a suitable dose of cells according to any one of claims 1 to 17, thereby improving at least one symptom.
